(19) United States
(12) Patent Application Publication
(10) Pub. No.: US 2024/0350821 A1
(43) Pub. Date: Oct. 24, 2024

(54) APPARATUS FOR TRANSCRANIAL MAGNETIC STIMULATION

(71) Applicant: The Regents of the University of California, Oakland, CA (US)

(72) Inventors: Peter Asbeck, Del Mar, CA (US); Mark Stambaugh, La Jolla, CA (US); Wing Ching Vincent Leung, Waco, TX (US); Milan Theodore Makale, San Diego, CA (US); Sravya Alluri, San Diego, CA (US)

(21) Appl. No.: 18/684,434

(22) PCT Filed: Aug. 19, 2022
(86) PCT No.: PCT/US2022/040948

§ 371 Date: Feb. 16, 2024

Related U.S. Application Data
(60) Provisional application No. 63/391,670, filed on Jul. 22, 2022.
(60) Provisional application No. 63/235,369, filed on Aug. 20, 2021.

(51) Int. Cl. A61N 2/00; A61N 2/02

(52) U.S. Cl. CPC A61N 2/006; A61N 2/02

(57) ABSTRACT

In some embodiments, there is provided a transcranial magnetic stimulation system, which comprises an inductor configured to be disposed on, or proximate to, a surface of a head to generate a current that induces an electric field through electromagnetic induction, wherein the inductor includes a first terminal and a second terminal; at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in a reversal in the current through the inductor and a voltage pulse to be generated across the inductor.

### CROSS REFERENCE TO RELATED APPLICATIONS

This application claims priority to U.S. Provisional Application No. 63/235,369 entitled “APPARATUS FOR TRANSCRANIAL MAGNETIC STIMULATION” and filed on Aug. 20, 2021, and U.S. Provisional Application No. 63/391,670, entitled “APPARATUS FOR TRANSCRANIAL MAGNETIC STIMULATION WITH IMPROVED PERFORMANCE” and filed on Jul. 22, 2022. The disclosures of the foregoing provisional applications are incorporated herein by reference in their entirety.

### TECHNICAL FIELD

This application relates generally to therapeutic applications of magnetic stimulation.

### BACKGROUND

Transcranial magnetic stimulation, or repetitive transcranial magnetic stimulation (rTMS), is a technique that can be used to treat a variety of things including neuropsychological conditions, depression, anxiety, addiction, autism, brain injury, and the like. However, many rTMS systems today are relatively large and expensive, and as such, are limited to use in hospitals and clinics.

### SUMMARY

In some example embodiments, there may be provided an apparatus for transcranial magnetic stimulation.

In some embodiments, there is provided a transcranial magnetic stimulation system, which comprises an inductor configured to be disposed on, or proximate to, a surface of a head to generate a current that induces an electric field through electromagnetic induction, wherein the inductor includes a first terminal and a second terminal; at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in a reversal in the current through the inductor and a voltage pulse to be generated across the inductor.

In some variations of the methods, systems, and computer program products, one or more of the following features can optionally be included in any feasible combination. While the switch is open, the reversal in current reverses at a rate that faster, when compared to a rate at which the current increases in the inductor while the switch is closed. The current reverses at the rate that is at least three times faster, when compared to the rate at which the current increases in the inductor while the switch is closed. The system may further include a fly back capacitor coupled to at one end to the first terminal of the inductor and at the other end to the second terminal of the inductor, wherein the energy sink path comprises the fly back capacitor. The energy sink path provides a path for a rapid transfer of the energy during the reversal in the current through the inductor, and wherein the rapid transfer is caused in part by the fly back capacitor in the energy sink path, the fly back capacitor having a capacitance that smaller than a capacitance of the at least one source capacitor. The capacitance of the fly back capacitor is at least nine times smaller than the capacitance of the at least one source capacitor. The system may further include a fly back resistor coupled to at one end to the second terminal of the inductor, wherein the energy sink path comprises the fly back resistor. The fly back resistor is further coupled to the first terminal of the inductor. The fly back resistor is further coupled to a fly back capacitor coupled to at one end to the first terminal of the inductor and at the other end to the second terminal of the inductor. The fly back capacitor is further coupled to a diode that is in parallel to the fly back capacitor. The at least one source capacitor is further coupled to a first terminal of the switch, and wherein the second terminal of the inductor is further coupled to a second terminal of the switch. The switch comprises at least one of: one or more insulated-gate bipolar transistors, one or more field effect transistors, and one or more metal-oxide-semiconductor field-effect transistor. The system may further comprise a switch controller coupled to the switch to close the switch to enable the current in the inductor to increase towards a threshold current amount and, in response to the threshold current amount through the inductor being reached, open the switch. The threshold current amount being reached is determined based on an expiry of a timer or based on measurement of the current through the inductor reaching the threshold current amount. The system may further comprise a diode coupled to the second terminal of the capacitor, the energy sink path, and a first terminal of the switch, wherein when the switch is open, the diode enables a portion of the reversal in current to be recycled into the at least one source capacitor. The system may further comprise a charging unit providing a direct current power source to the at least one capacitor during a charging phase of the at least one capacitor.

In some embodiments, there is provided a transcranial magnetic stimulation system comprising an inductor configured to be disposed on or proximate to a surface of a head to generate a current that induces an electric field through electromagnetic induction, wherein the inductor includes a first terminal and a second terminal; at least one source capacitor coupled to at least the first terminal of the inductor; and a switch configured to at least: close to discharge the at least one source capacitor towards the inductor; and open, in response to a threshold current amount through the inductor being reached, wherein energy from the inductor is transferred to an energy sink path thereby resulting in a drop in inductor current towards zero at a rate that is faster, when compared to a rate at which the current increases in the inductor while the switch is closed.

In some variations of the methods, systems, and computer program products, one or more of the following features can optionally be included in any feasible combination. The current drops at the rate that is at least three times faster, when compared to the rate at which the current increases in the inductor while the switch is closed. The energy sink path comprises at least one resistor coupled to the second terminal of the inductor.

In some embodiments, there is provided a method comprising: placing an applicator including an inductor disposed on, or proximate to, a surface of a head, wherein the inductor generates a current that induces an electric field through electromagnetic induction, wherein the inductor includes a first terminal and a second terminal; and initiating transcranial magnetic stimulation, wherein the inductor is comprised in a transcranial magnetic stimulation system, wherein the transcranial magnetic stimulation system comprises: at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in at least one of: a reversal in the current through the inductor and a voltage pulse to be generated across the inductor or a drop in inductor current towards zero at a rate that is faster, when compared to a rate at which the current increases in the inductor while the switch is closed.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts an example of an rTMS system, in accordance with some example embodiments;

FIG. 1B depicts an example implementation of the pulse generation circuit, in accordance with some embodiments;

FIG. 1C depicts an example of an inductor, in accordance with some embodiments;

FIG. 1D depicts another example of an inductor, in accordance with some embodiments;

FIGS. 1E, 1F, and 1G depicts examples of pulse generation circuits, in accordance with some embodiments;

FIG. 2 depicts various plots associated with a simulation of a pulse generation circuit, in accordance with some example embodiments;

FIG. 3 compares the biphasic pulses of conventional rTMS systems with the monophasic pulses generated by the pulse generation circuit, in accordance with some example embodiment;

FIG. 4 depicts another example of a pulse generation circuit, in accordance with some embodiments;

FIG. 5 depicts a plot of the energy per pulse as a function of the product of peak pulse electric field and pulse duration, in accordance with some embodiments;

FIG. 6 depicts a plot of the electric field measurements versus distance to the bottom of the inductor, in accordance with some embodiments;

FIG. 7 depicts plots of a representative pattern of E field intensity according to the simulations, in accordance with some embodiments;

FIG. 8 depicts plots of measured magnetic pulses at various distances, in accordance with some example embodiments; and

FIG. 9 depicts an example block diagram of a system, in accordance with some example embodiments.

### DETAILS DESCRIPTION

As noted above, repetitive transcranial magnetic stimulation (rTMS) systems tend to be relatively large and expensive, so as to be limited to use in hospitals and clinics. As such, there is a need for more compact, lightweight, and/or affordable rTMS systems that could enable treatment of patients across a wider variety of treatment settings (e.g., small clinics and home settings) and/or more frequently (e.g., daily basis). To provide such a compact rTMS system, there are issues related to power, such as power dissipation, battery requirements, size and/or weight of the components, and safety considerations.

The effectiveness of an rTMS system in eliciting a neural response is related to the electric field that is generated in the cortex at the neuron locations being activated. This electric field is related to (or caused by) the rate of change of the magnetic field produced by the rTMS system and, in particular, rTMS system's inductor(s) (also referred to as coils) where the magnetic field is produced by repetitive current pulses flowing through the inductor placed in the vicinity of the patient's head, for example.

In some embodiments, the rTMS system disclosed herein includes a pulse generation circuit that generates a boosted electric field, such that the rTMS system can be implemented with decreased size and/or power requirements while maintaining the performance parameters of rTMS treatment. In an implementation for example, the rTMS system (also referred to as a boost rTMS system) consistent with the disclosure herein was implemented in a compact circuit implementation for which an electric field of 300 Volts/meter (V/m) at a depth of 1.5 centimeters (cm) (e.g., from the edge of the inductor/coil) was measured using a power supply voltage of 300 V, and an effective pulse duration (half amplitude width) of 23 μsec. This field intensity may be on the order of (e.g., about) 1.8-2 times the corresponding maximum electric field intensities in commercial systems at full power and comparable depths, which utilize pulse widths in the range 70-280 μsec, and power supply voltages of 1800-3000V. The boost rTMS system disclosed herein may provide an electric field boost of 4 times (4×), relative to that obtainable from the power supply voltage, although electric field boost in the range of 2 to 6 times may also be realized. The boost rTMS system's reduced power supply and the high generated electric field may thus lead to improvements in power efficiency, enhanced safety, and lower voltage ratings needed for various components to reduce cost and enhance portability. Although the previous example provides specific values, these are for purposes of illustration and other values may be implemented within the scope of the disclosure.

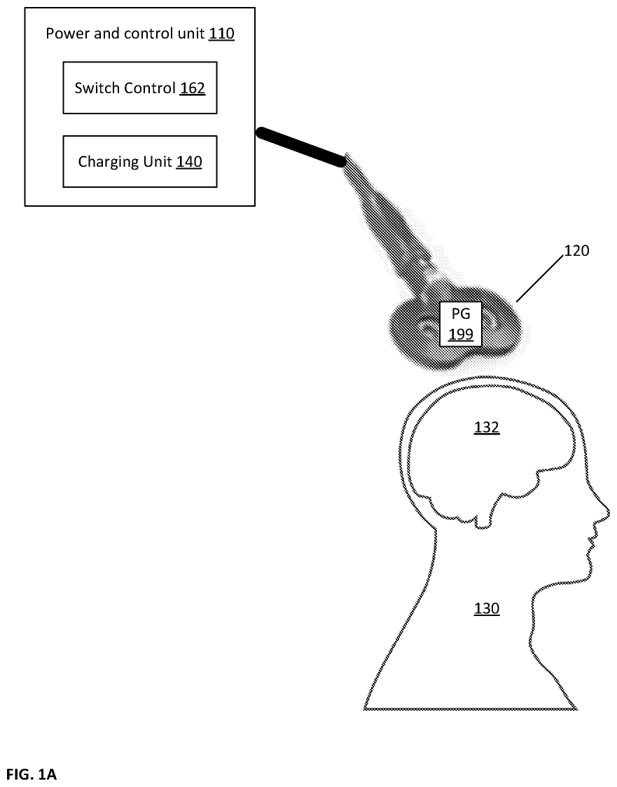

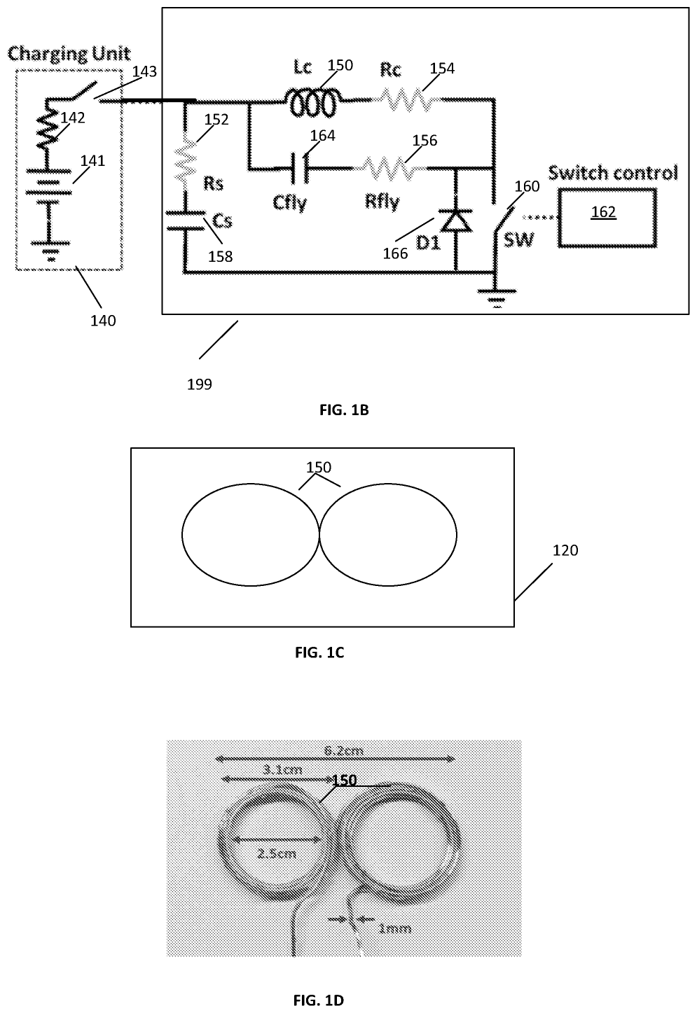

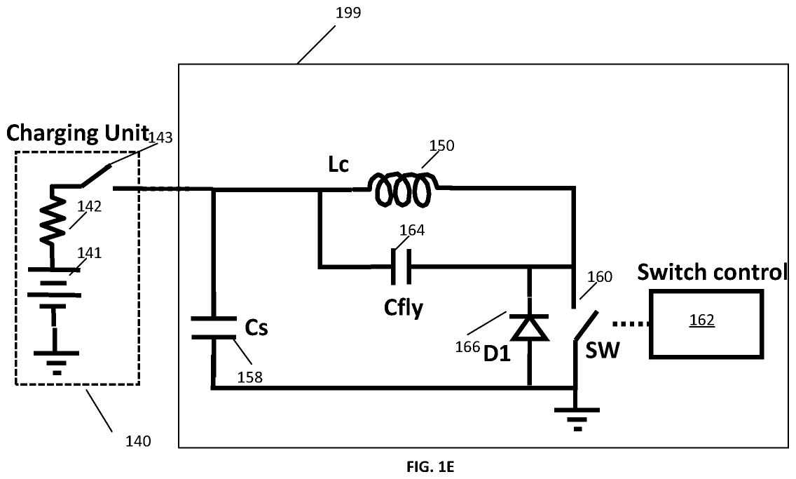

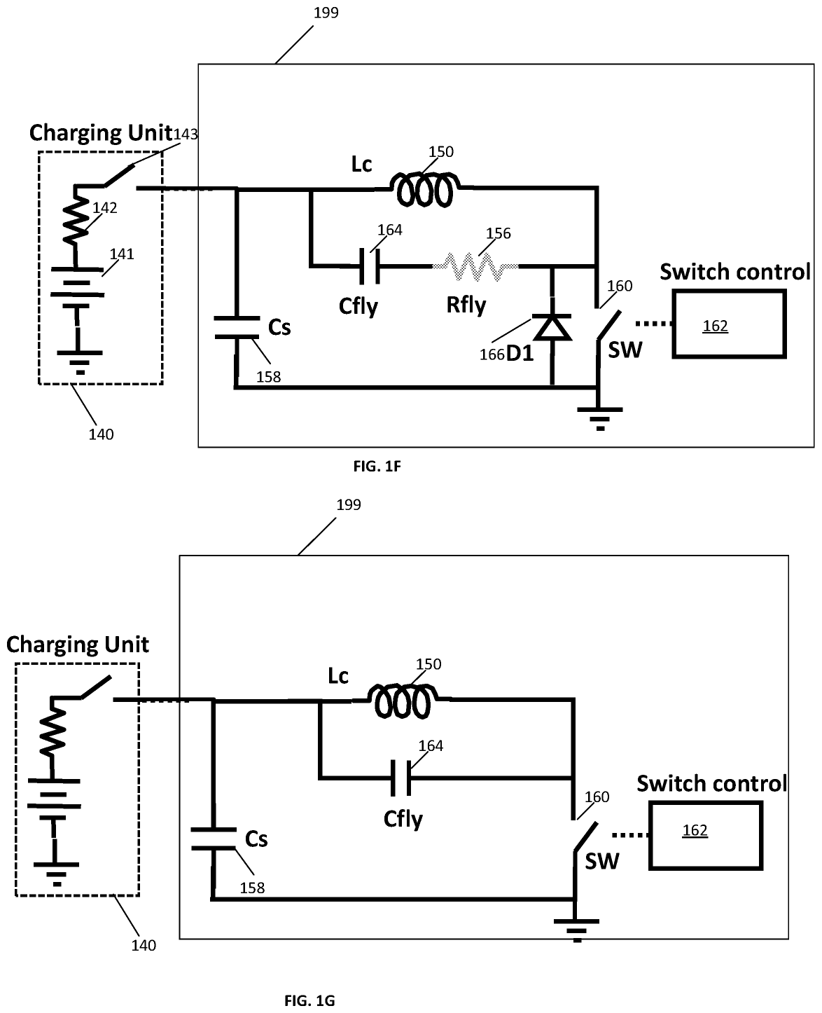

FIG. 1A depicts an example of an rTMS system including a power and control unit 110 that may be used to control and/or power an applicator, such as a wand 120, which is coupled to the power and control unit. The applicator, such as the wand, may include an inductor (also referred to as coil) that generates a magnetic pulse (or, e.g., electric or electromagnetic field) that stimulates nerve cells in for example the cortex 132. The power and control unit may further include a switch control unit 162 that is configured to control the magnetic pulses that emanate from the inductor towards the cortex of the user. And, the power and control unit may include a charging unit 140 that provides power to a pulse generation circuit 199.

In operation, a user holding the wand 120 places the wand on, or proximate to, a patient 130 and, in particular, on or proximate to tissue being treated with rTMS. In the example, the wand 120 is being placed proximate to the cortex (e.g., outer layer) of the brain 132, although other types of tissue may benefit from the application of the rTMS. For example, a user of the rTMS system may depress a switch on the wand 120 or the power and control unit 110, such that the switch causes a pulse generation circuit to generate, using an inductor in the wand, voltage pulses that cause an electro (magnetic) field. This field induces neuronal activity within the tissue of the cortex.

Although the previous example refers to the applicator as a wand, the applicator may take other forms as well, such as a skull cap, bandage, and/or other medium configured to contain at least the inductor (or coil) and, in some implementations, at least a portion of the pulse generation circuit.

It is believed that the interaction of rTMS system with the cortex is via induced electric fields, and that in order to be effective, the rTMS system should produce an electric field above a threshold amount for neuronal excitation. The threshold electric field may be dependent on a number of factors, such as pulse duration and wave shape, axon orientation, and type of nerve and axon orientation, but in some applications of rTMS the threshold electric field may be in the range of 60-100 Volts/meter (V/m) for some pulses. This electric field may need to be maintained for a given amount of time. It is disclosed herein that the energy required to produce an electric field Eₚ pulse of duration tₚ is proportional to (Eₚ tₚ)², noting that a portion of this energy may be recycled by the pulse generation circuit. For a compact rTMS system as disclosed herein, the pulse duration tₚ can be controlled to a minimum allowable value (e.g., 5 microseconds (μs) and extended to above 25 μs) and/or to optimize the possible tradeoffs between Eₚ and tₚ while providing the desired treatment effects for a given product Eₚ tₚ.

In some embodiments, there is provided a pulse generation circuit, such as the pulse generation circuit 199, that can generate pulses in accordance with a given pulse duration, such as pulse duration tₚ. The electric field(s) generated with short pulse durations (e.g., tₚ on the order of 10 μs) have correspondingly higher amplitudes, when compared to the amplitudes produced with longer duration pulses used by a conventional rTMS systems.

In some embodiments, the pulse generation circuit 199 disclosed herein may be configured to provide short pulse durations (e.g., short magnetic or electromagnetic pulses) and correspondingly higher amplitudes, which are generated using an amplitude “boost” factor on the order of 4-6 times, for example. The basis for achieving higher electric field E during a shorter time follows directly from the fact that E is proportional to dB/dt, where B is the magnetic flux density. A magnetic field is set up by a current I in the rTMS inductor (e.g., coil), and then rapidly dropped to zero, leading to a high value of dB/dt. Moreover, as pulses with short duration (e.g., less than 50 μsec) can be effective in producing physiological effects during rTMS application, and the use of short pulses and control over current waveforms can lead to reduced energy requirements.

FIG. 1B depicts an example implementation of the pulse generation circuit 199, in accordance with some embodiments. The pulse generation circuit 199 is configured to provide the noted short pulse durations and correspondingly higher amplitudes, when compared to conventions rTMS systems.

The pulse generation circuit 99 may be coupled to the charging unit 140, which provides a source of power to charge the source capacitor Cs 158. For example, the charging unit 140 may include a direct current (DC) power source 141 or other type of source of power that can charge the source capacitor 158 to an initial characteristic voltage Vo. The charging unit may include one or more other components, such as a resistor 142, a switch 143, and/or other components (e.g., capacitors, etc.). The charging unit is used during a source capacitor 158 charging phase. During the source capacitor charging phase, the switch 143 is closed (and switch 160 is in an open state) to allow the charging unit 140 to charge the source capacitor 158 to a voltage such as an initial or characteristic voltage Vo.

In the example of FIG. 1B, the inductor 150 (labeled Lc) may be included in (e.g., contained in or placed on the exterior surface of) an applicator, such as the wand 120, although the inductor 150 may be included in other types of devices as noted above to enable application of rTMS to the user 130. To illustrate further, pulsed electric (and/or magnetic) fields are generated by creating short duration current pulses through the inductor 150 (which is also referred to herein as a coil). For example, a magnetic field is set up by a current I in the inductor 150 and then the current is rapidly dropped to zero. This rapid drop leads to a high value of dB/dt (i.e., rate of change of the amplitude of the magnetic field).

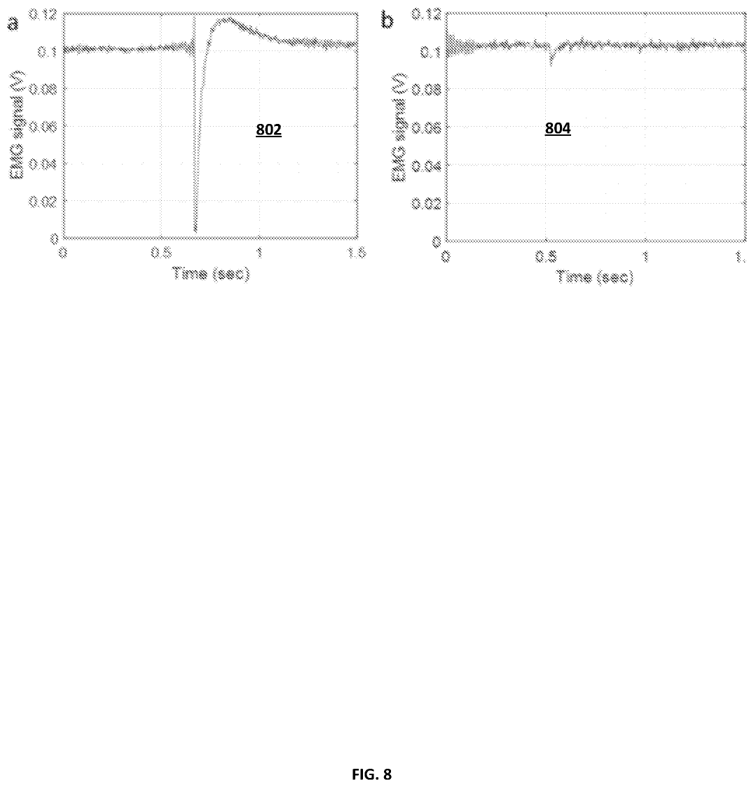

Although FIG. 1B depicts a single inductor, a plurality of inductor may be implemented at 150 as well. In some embodiments, the inductor comprises a FIG. 8 shaped coil 150 included within (e.g., contained within, affixed to the surface of, etc.) the wand 120 as shown at FIG. 1C. However, the coil 150 may take other shapes as well (e.g., circular). Moreover, the coil 150 may include one winding or more windings as well. FIG. 1D depicts another example of the coil 150 (which may be included with the wand or other type of applicator), although the dimensions noted for the inductor are merely an example as other values may be used as well.

Referring again to FIG. 1B, the resistors Rs 152, Rc 154, and Rfly 156 represent parasitic resistors (or resistance) that accompany the physical circuit implementation. FIG. 1E is similar in operation to FIG. 1B but omits the resistors Rs 152, Rc 154, and Rfly (or Rflyback) 156.

Referring to FIGS. 1B and 1E, in operation, the source capacitor Cs 158 is initially charged by the charging unit 140 during the source capacitor 158 charging phase. While in the source capacitor charging phase, the switch 143 is closed (and switch 160 is opened) to allow the charging unit 140 to charge the source capacitor 158 to the initial characteristic voltage Vo. The switch control unit 162 may be coupled to the switch 143 to enable closing the switch 143, and the switch control unit 162 may be coupled to switch 160 to enable opening switch 160. When the source capacitor is charged to its initial characteristic voltage Vo, the charging is complete and the switch 143 can be opened and the pulse generation phase can begin.

During the pulse generation phase, the switch 160 changes state from an open to a closed state, so current pulses originate through the discharge of the capacitor ('s 158 into the inductor 150, which generates a magnetic field. After the current Ic (which is the current of inductor Lc 150) builds to a predetermined current value (e.g., +I_{MAX}), the switch 160 is opened. The switch controller 162 may trigger the switch 160 to open in response to Ic reaching the predetermined current value (or threshold current value). For example, the switch controller 162 may monitor the current in the inductor 150 (Ic) and when Ic reaches the threshold current level, the switch controller may open switch 160. Alternatively, or additionally, the switch controller may use a timer (e.g., timing circuit or other type of timer), such that the expiry of the timer corresponds to the threshold current level at the inductor 150. Alternatively, or additionally, the switch controller may measure the current through the inductor to determine when it reaches the threshold current level. Alternatively, or additionally, the threshold current level may be adjusted to increase or decrease the electric field generated by the inductor 150.

When the switch 160 is closed as noted, a magnetic field is set up by a current Ic in the inductor 150. But when the switch 160 opens again in response to the inductor's 150 current reaching a predetermined (or threshold) current amount, the opening of switch 160 causes (1) the current Ic in the inductor 150 to rapidly drop as the current in the inductor (or energy) moves toward fly back capacitor 164 and (2) the voltage across the inductor 150 (and, e.g., across capacitor 164) to rapidly rise to a high amplitude (as well as cause a high rate of change of the amplitude of the magnetic field). Here, the end effect is a short duration pulse (e.g., between 20 μsec and 50 μsec, although other values may be realized) of magnetic (or electromagnetic energy) from the inductor 150 into the subject 130, for example.

In some embodiments, the rate of current build up in the inductor 150 (e.g., when switch 160 is open) is about 3 times slower than the rate of current discharge (e.g., when switch 160 is open) though the fly back portion of the circuit, such as fly back capacitor 164. For example, when the switch 160 opens, the reversal in inductor current is at a rate that is at least more than three times faster than the rate at which the current in the inductor is initially built-up while the switch 160 is closed. In this example, the fly back capacitor 164 would have a capacitance that is at least nine times smaller than a capacitance of the source capacitor 158 (see, e.g., equation 5 below). Alternatively, or additionally, when the switch 160 opens, the reversal in inductor current is at a rate that is at least more than four times faster than the rate at which the current in the inductor is initially built-up while the switch 160 is closed, and in this example, this rapid drop is due in part to the fly back capacitor 164 being at least sixteen times smaller than a capacitance of the source capacitor 158.

As noted, the fly back capacitor, Cfly (or Cflyback) 164 may be coupled in parallel (or across) the inductor 150 to enable the rapid drop in inductor current (or, e.g., rapid transfer of energy) into the capacitor 164. When the switch 160 is opened again, the inductor's 150 current flows into Cfly 160, whose voltage Vfly (or Vflyback) builds up rapidly due to the rapid inflow of current. When the initial inductor current Ic drops to zero as a result of the switch 160 opening again, the voltage Vfly (which is across the capacitor 164) causes a reverse current to flow in the inductor 150. This rapid change in the inductor current from a forward direction to a reverse direction generates a large voltage across the inductor 150. This large voltage across inductor 150 provides a correspondingly large electric field in the region (or space) proximate to the inductor 150. And, the large voltage across inductor and correspondingly large electric field are proportional to the time derivative of the coil current dI_{c}/dt.

The reverse current through the inductor 150 may have a duration comparable to that of the discharge of the forward current through the inductor (which can lead to an unwanted oscillatory behavior of the Lc and Cfly resonance). However, this unwanted resonance may be reduced or largely eliminated by the turn-on of a diode D1 166, which parallels the switch 166 to the main capacitor Cs 158. The subsequent inductor current delivers a charge back to the capacitor Cs 158, which may provide recycling of a portion of the energy delivered to the inductor 150. A small fraction of the current may remain in the Lc-Cfly resonator, which may cause small amplitude ripples of current and voltage subsequent to the main pulse.

The inductor's 150 voltage Vmax (which is reached during the noted rapid change of the inductor's 150 current) may be determined based on the ratio Cs 158 to Cfly 164. This voltage Vmax can be boosted over the characteristic voltage Vo (which is used to charge Cs) by about 2 to 10 times, for example. This boost is referred to as a boost factor. There is a corresponding electric field (also referred to as an electromagnetic or magnetic field) generated by the inductor 150. This field is proportional to the inductor's voltage and is enhanced by the boost factor for a given inductor relative to what can be obtained by only the direct application of the characteristic voltage Vo.

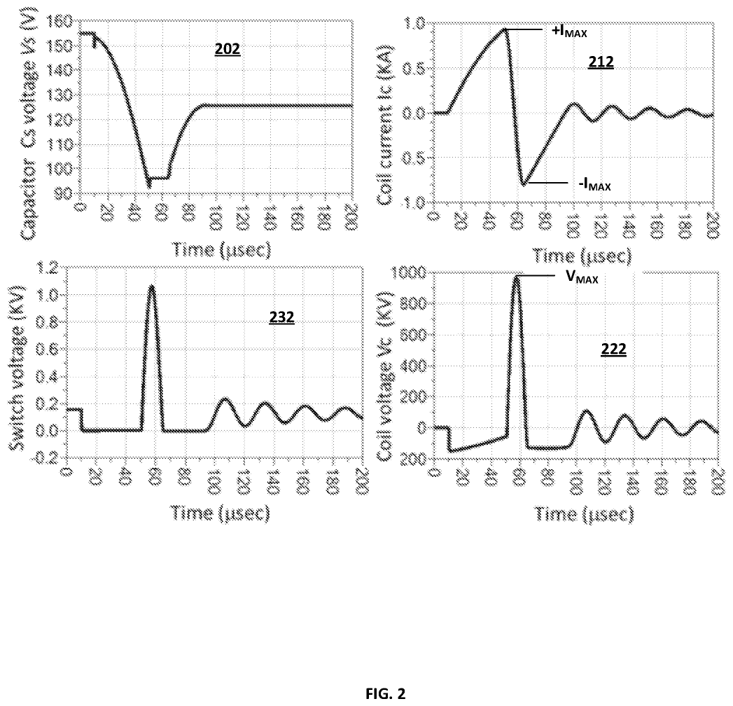

FIG. 2 depicts various plots 202, 212, 222, and 232 associated with a simulation of the pulse generation circuit 199. For the simulation depicted in these plots, the characteristic voltage Vo (to which the Cs 158 is charged) is 155V, the value of Cs 158 is 380 μF, and the value of Cfly 164 is 3.8 μF (although these values are merely examples for purposes of the simulation so other values may be implemented at circuit 199).

When the switch 160 closes (at time t=10 μsec as shown at plot 232), the inductor or coil's 150 current Ic builds up to a value of 950A (see +I_{MAX} at plot 212) as the capacitor ('s discharges from 155V to 95V as can be seen at plot 202. When the switch 160 subsequently opens again at t=50 μsec, the inductor 150 current Ic drops rapidly and reaches a value of −900 A (see −I_{MAX} at plot 212) and the voltage Vc across the inductor 150 changes from −155V and rapidly builds to almost 1000V (see V_{MAX} at plot 222) during the large current transient. To illustrate further, the electric field at the site of the neurons produced by a given inductor with a given current waveform is proportional to the voltage across the inductor per turn of the inductor, so the inductor voltage considerations and measurements are a good proxy for electric field evaluation at the neuron locations.

In the example of plot 212, the initial build-up of the current at 10 μseconds to 50 μseconds is slower (e.g., at least 4 times slower), when compared to the rapid out flow of the current between +Imax and −Imax (e.g., between about 50 μseconds and 62 μseconds). For example, when the switch 160 opens, the reversal in inductor current is at a rate that is at least more than three times faster than the rate at which the current in the inductor is initially built-up while the switch 160 is closed. In this example, this rapid drop is due in part to the fly back capacitor 164 being at least nine times smaller than a capacitance of the source capacitor 158. Alternatively, or additionally, the reversal in inductor current is at a rate that is at least more than two times faster than the rate at which the current in the inductor is initially built-up while the switch 160 is closed, and in this example, the capacitor 164 being at least four times smaller than a capacitance of the source capacitor 158 (see, e.g., equation 5 below). Alternatively, or additionally, the reversal in inductor current is at a rate that is at least more than four times faster than the rate at which the current in the inductor is initially built-up while the switch 160 is closed, and in this example, the capacitor 164 being at least 16 times smaller than a capacitance of the source capacitor 158 (see, e.g., equation 5 below). Alternatively, or additionally, the reversal in inductor current is at a rate that is at least more than five times faster than the rate at which the current in the inductor is initially built-up while the switch 160 is closed, and in this example, the capacitor 164 being at least 25 times smaller than a capacitance of the source capacitor 158 (see, e.g., equation 5 below). Alternatively, or additionally, the reversal in inductor current is at a rate that is at least more than six times faster than the rate at which the current in the inductor is initially built-up while the switch 160 is closed, and in this example, the capacitor 164 being at least 36 times smaller than a capacitance of the source capacitor 158 (see, e.g., equation 5 below). Alternatively, or additionally, the reversal in inductor current is at a rate that is at least more than seven times faster than the rate at which the current in the inductor is initially built-up while the switch 160 is closed, and in this example, the capacitor 164 being at least 49 times smaller than a capacitance of the source capacitor 158 (see, e.g., equation 5 below). Alternatively, or additionally, the reversal in inductor current is at a rate that is at least more than eight times faster than the rate at which the current in the inductor is initially built-up while the switch 160 is closed, and in this example, the capacitor 164 being at least 64 times smaller than a capacitance of the source capacitor 158 (see, e.g., equation 5 below). Alternatively, or additionally, the reversal in inductor current is at a rate that is at least more than nine times faster than the rate at which the current in the inductor is initially built-up while the switch 160 is closed, and in this example, the capacitor 164 being at least 81 times smaller than a capacitance of the source capacitor 158 (see, e.g., equation 5 below). Alternatively, or additionally, the reversal in inductor current is at a rate that is at least more than 10 times faster than the rate at which the current in the inductor is initially built-up while the switch 160 is closed, and in this example, the capacitor 164 being at least 100 times smaller than a capacitance of the source capacitor 158 (see, e.g., equation 5 below).

At 222, the large transient inductor voltage Vc is opposite in sign to the initial applied voltage Vo (Vo and Vc are defined here with signs shown in FIG. 1B). In this example, the large transient coil voltage Vc has a duration 14 μsec in the example shown. With the subsequent turn-on of diode D1 166 (caused by the opening of switch 160), the inductor 150 current recharges the capacitor Cs 158, leading to an increase of its voltage to 125V as shown at plot 222 (thus showing the recycling provided by the fly back portion of the circuit (e.g., Cfly 164). At this stage, the circuit 199 can again enter the source capacitor charging phase where the switch 143 is closed (and switch 160 is kept opened), so that the source capacitor 158 can charge to the initial characteristic voltage Vo before resuming the pulse generation phase described above.

Quantitative analysis of the operation of the pulse generation circuit 199 circuit resembles the simulated results of FIG. 2. During the initial phase of operation, the charging of inductor 150 Lc is governed by the transient behavior of the Lc-Cs resonator with resonant frequency f₁ and coil current and voltage given by

$${f}_{1} - {( Lc - Cs )}^{1 / 2} / 2 π \\ \text{?} ( t ) - {I}_{cmax 1} sin ( 2 π {f}_{1} t ) \\ \text{?} ( t ) - 2 π {f}_{1} Lc {I}_{cmax 1} cos ( 2 π {f}_{1} t ) \qquad ( 1 )$$

$$\text{?} \text{indicates text missing or illegible when filed}$$

The duration of the charging pulse may be chosen to slightly below a quarter (¼) of the resonant period, before the current reaches its peak value

$${I}_{cmax 1} - Vo / 2 π {f}_{1} Lc \qquad ( 2 )$$

When the switch SW 160 opens, the values of inductor 150 current Ic and inductor 150 voltage Vc follow the behavior of the Lc-Cfly resonator (150-164), with higher resonant frequency f₂ and time dependence of Vc and Ic is then given by

$${f}_{2} - {( Lc - Cfly )}^{1 / 2} / 2 π \\ Lc ( t ) - {I}_{cmax 2} cos ( 2 π {f}_{2} t ) \\ Vc ( t ) - 2 π {f}_{2} Lc {I}_{cmax 1} sin ( 2 π {f}_{2} t ) \qquad ( 3 )$$

The duration Tp of the high voltage pulse (measured between start and ending times) is close to half (½) of the resonant period,

$$Tp - 1 / 2 {f}_{2} - {π ( LcCfly )}^{1 / 2} \qquad ( 4 )$$

The maximum inductor voltage V_{cmax} during the phase when the switch 160 is open is estimated by equating I_{cmax1} and _{Icmax2}, which leads to

$${V}_{cmax 2} / Vo - {f}_{2} / {f}_{1} - ( Cs / Cfly ) \text{?} \qquad ( 5 )$$

$$\text{?} \text{indicates text missing or illegible when filed}$$

for the case where the charging pulse duration is chosen to be exactly ¼ of the f₁ resonant period. The energy Eₚ that is dissipated during the pulse can be estimated from the initial and final voltages (Vo and Vof) across the capacitor Cs following

$$Ep - 1 / 2 Cs ( {Vo}^{2} - Vof ) \qquad ( 6 )$$

The dissipation (which is associated with the parasitic resistances in the current flow path) is due to the inductor 150, switch, and (to a lesser extent) to the capacitors and to the mutual inductance coupling between the inductor 150 and neighboring resistive materials, including the subject's 130 body. The resistive dissipation per unit time is proportional to the square of the current flowing and thus resistive dissipation per unit time is of interest to minimize both the duration of the current and its amplitude, as well as the component resistances. The fact that the inductor voltage and induced electric field follow dIc/dt as the current changes from Imax to approximately −Imax reduces by ¼ the dissipation expected for changes from Imax to 0 with a similar duration and dIc/dt.

FIG. 1F depicts another example of the pulse generation circuit, in accordance with some embodiments. FIG. 1F is similar to FIG. 1E in some respects but includes the addition of Rfly 156. In the example of FIG. 1F, Rfly 156 is a resistor (which may be a variable resistor) that can be used to control the pulse width generated by the inductor 150.

FIG. 1G depicts another example of the pulse generation circuit, in accordance with some embodiments. FIG. 1G is similar to FIG. 1E in some respects but omits the diode 166. With the omission of the diode, the voltage generated across the inductor 150 may have additional ripple.

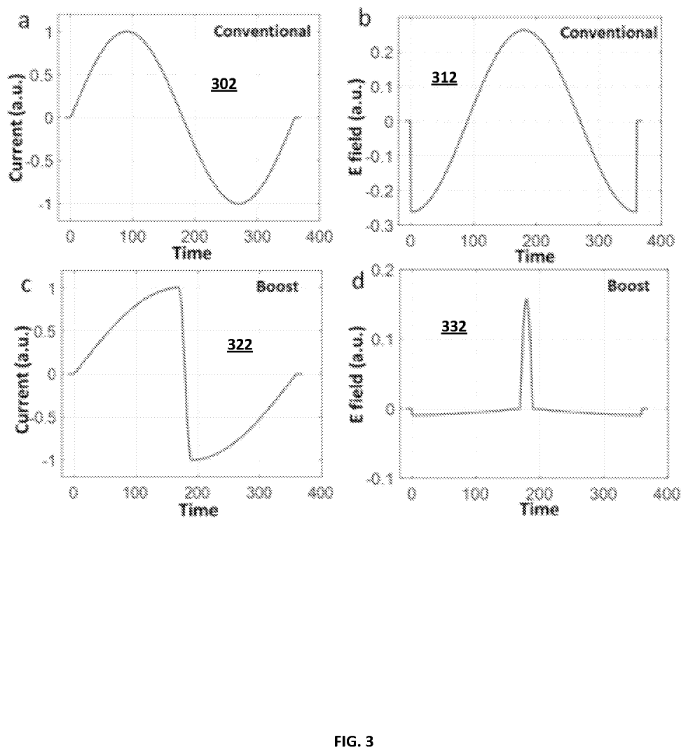

FIG. 3 depicts plots of the electric field waveforms formed by the inductor 150, in accordance with some embodiments. The electric fields induced with the pulse generation circuit 199 can differ from the biphasic waveforms used in conventional rTMS systems as shown in FIG. 3, where plots a 302 and b 312 depict the conventional rTMS biphasic waveforms of the coil current and the induced electric field and plots c 322 and d 332 depict the waveforms of the coil current and the induced electric field for the monophasic pulse generation circuit disclosed herein. For example, a difference is that the conventional biphasic electric field pulse achieves lower amplitude for a given input voltage; the pulse of the pulse generation circuit 199 is increased by the boost factor in the range 4-10.

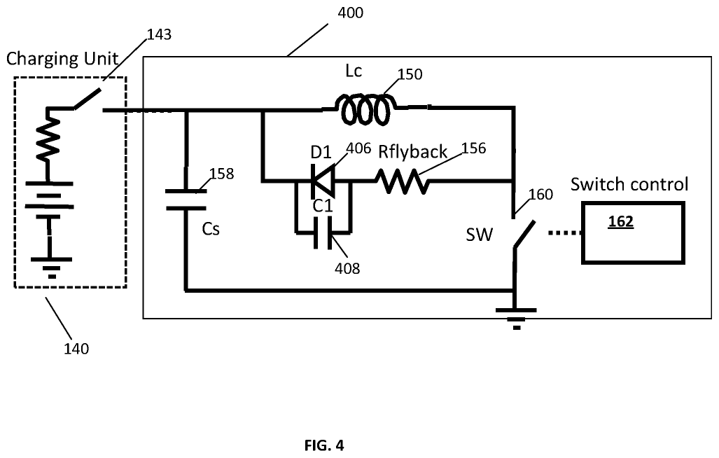

FIG. 4 depicts another example of a pulse generation circuit 400, in accordance with some embodiments. The pulse generation circuit 400 is similar in some respects to pulse generation circuit 199 but shows a fly back resistor 156 (which in this case is a physical resistor which may be a variable resistor) in series with the capacitor 408. The inductor 150 needs a fly back path (e.g., resistor 156 and capacitor 408) to enable the rapid drop or transfer of energy into the fly back path. In this example, the diode 406 is coupled in parallel to capacitor.

The pulse generation circuit 400 may be coupled to the charging unit 140, which provides a source of power to charge the source capacitor Cs 158. Like circuit 199, during the source capacitor 158 charging phase, the switch 143 is closed (and switch 160 is opened) to allow the charging unit 140 to charge the source capacitor 158 to an initial characteristic voltage Vo.

In operation, the source capacitor Cs 158 is initially charged by the charging unit 140 during the source capacitor 158 charging phase. While in the source capacitor charging phase, the switch 143 is closed (and switch 160 is opened) to allow the charging unit 140 to charge the source capacitor 158 to the initial characteristic voltage Vo. When the source capacitor is charged to its initial characteristic voltage Vo, the charging is complete and the switch 143 can be opened by the switch control unit 162, such that the pulse generation phase can be initiated. During the pulse generation phase, the switch 160 is closed so current pulses originate through the discharge of the capacitor Cs 158 into the inductor 150. After the current Ic builds to a predetermined current value (which can be determined by using for example a timer, such that the expiry of the timer corresponds to the predetermined or threshold current value level at the inductor 150), the switch 160 is opened. When the switch 160 opens in response to the current Ic reaching a predetermined current value, this causes the current Ic in the inductor 150 to rapidly drop and flow through to the fly back portion of the circuit Rflyback 156, capacitor 408) and the voltage across the inductor 150 to rapidly rise to a high amplitude (as well as a high rate of change of the amplitude of the magnetic field). Here, the end effect is a magnetic pulse from the inductor 150 into the subject 130, for example.

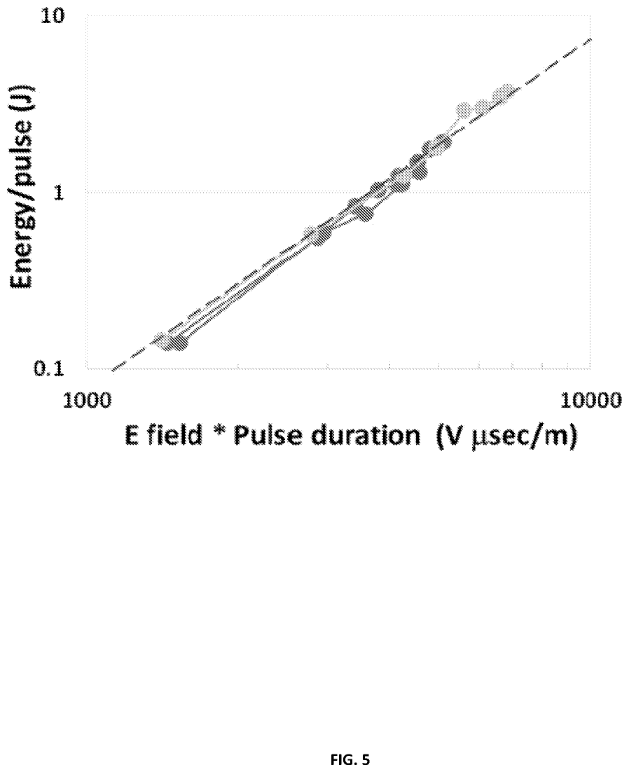

With respect to the energy Ep expended to produce a pulse electric field at the inductor 150, this energy Ep was evaluated from initial and final capacitor voltages. FIG. 5 depicts the energy per pulse as a function of the product of peak pulse electric field and pulse duration. Results are shown for several values of Cfly and Vo, with a fixed coil and a constant 1.5 cm distance for E field measurement. The quadratic relationship is expected based on simple approximate relationships:

$$Ep - 1 / 2 α Lc I {max}^{2} \\ Vc ( t ) - Lc dIc / dt \\ I max - β Vc max Tp / Lc$$

where Lc Imax² represents the energy stored in the coil and the factor a corresponds to the fraction of energy not recycled; Vcmax is the peak coil voltage during the pulse, and β is a factor of order unity characteristic of the pulse shape; Emax is the peak electric field, and y is a factor which relates the electric field at a given position to the overall coil voltage.

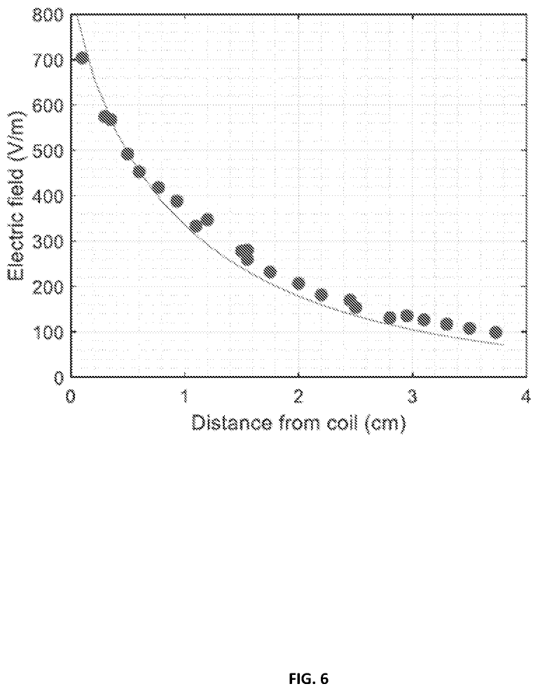

To characterize the attainable electric field as a function of distance from the coil, measurements made in air using an E field probe. FIG. 6 depicts a plot of the electric field measurements versus distance to the bottom of the inductor. The measured values are compared with the E field expected on the basis of numerical simulations, using

$$A ( r ) = \frac{µ}{4 π} \int \frac{I ( {r}^{′} ) \text{?}}{\vert r - {r}^{′} \vert} E = \frac{\partial A}{\partial t} B = ∇ x A$$

$$\text{?} \text{indicates text missing or illegible when filed}$$

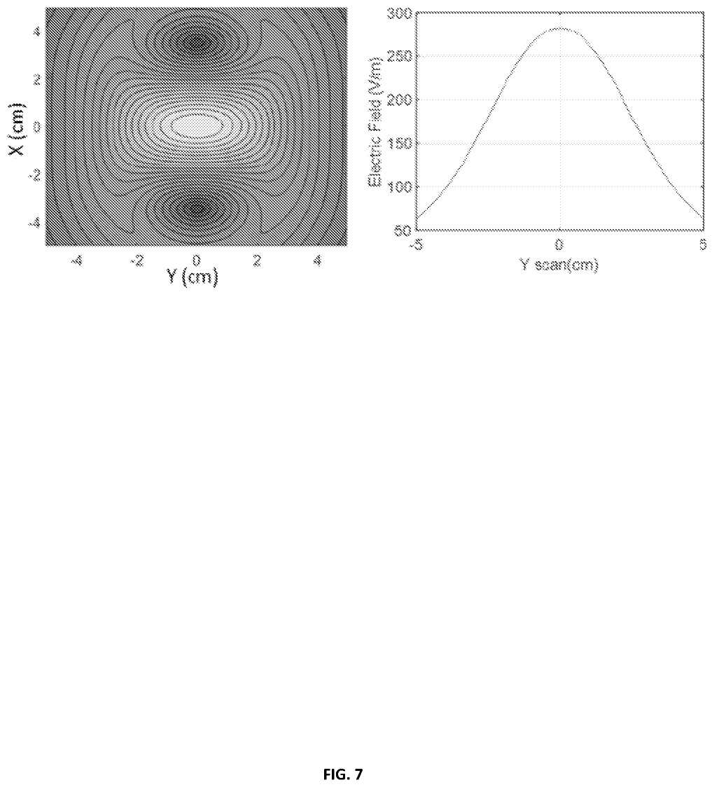

To evaluate the peak time derivative of Ic, Imax/πTp is used from the above analysis. A representative pattern of E field intensity according to the simulations is depicted at FIG. 7 (which in the example of FIG. 7 is at a distance of 1.5 cm from the inductor).

To verify the ability of the induced E field pulses to stimulate neural activity, pulses are applied to volunteer human subject 130 to excite the ulnar nerve (which originates from the brachial plexus and travels down arm) and to confirm motor responses from visual observation of the finger, thumb, and/or hand motion (caused by excitation of the ulnar nerves by the pulses from the inductor 150, for example). For example, a pulse widths of 23 μsec can be used, although other pulse widths may be applied as well. Moreover, the pulses can be applied successively (e.g., with a repetition rate of 10 pulses per second) in a burst lasting approximately 200 msec (with, for example, pulse amplitude decaying progressively for long burst times because of gradual discharge of capacitor ('s). In this example, the excitation was typically applied at the ventrolateral side of the forearm near the wrist, and motion of the fingers and/or thumb was observed. Male and female volunteers were tested, and motor responses were observed in all of the subjects. The input voltage level Vo for first observation of the motor response varied among the subjects, with a minimum of approximately 200V to about 280V. The maximum voltage used in our experiments was limited by the possibility of exceeding ratings of some components to 300V.

To verify that the circuit elicited a muscle neural response, electromyograpy (EMG) measurements were performed, using electrodes applied to the fingers (abductor digiti minimi). The coil was placed in close proximity to the ulnar nerve near the elbow, which provided a suitable distance from the EMG electrodes to minimize direct pickup of interfering electromagnetic signals. The coil was held at a distance of approximately 2 mm from the subject's skin, in order to eliminate the possibility of mechanical, rather than electromagnetic, excitation of the ulnar nerve. EMG signals were processed with a succession of low and high pass filters, digitized, and 60 Hz interference was removed digitally. A representative response to a single magnetic pulse is shown in FIG. 8 at plot a 802. For comparison, FIG. 8 at plot b 804 shows a pickup signal when the coil was moved away from the elbow by 4 cm, while maintaining the same distance from the EMG electrodes, showing the low level of electromagnetic interference.

With respect to cortex tissue, the neural systems is sensitive or responsive to the short pulses generated with for example the pulse generation circuit 199 or 400. The energy per pulse is approximately a function of the product of E field amplitude and pulse duration, so that an increase in amplitude by factor k that is accompanied by a decrease in Tp by the same factor k will leave the energy unchanged. The relationship between threshold excitation amplitude and pulse width for neuronal responses has been studied by Peterchev et al. for brain cortical stimulation in an rTMS configuration (Peterchev et al 2013). Peterchev et al. reported that a decrease in TMS pulse width leads to an increase in the TMS pulse amplitude at the threshold for motor evoked potential, along with a decrease in pulse energy. Using accurately shaped pulses, it was found that for pulse widths of 30, 60 and 120 μsec, the log-average values of motor threshold were 75.8%, 47.1% and 34.7% of the maximum amplitude. The corresponding products of pulse width and threshold amplitude for the pulse widths 30, 60 and 120 μsec are thus in the ratio 0.55, 0.68 and 1, indicating that a reduced value of the Tp Emax parameter occurs for the shorter pulses, in keeping with the reduced energy requirement per pulse. These results may be dependent on the specific pulse shapes involved, however, and the results for the pulsed waveforms obtained in the present work are unknown.

Furthermore, Rapp et al. (2022) working with a peripheral nerve-figure-8 coil model and a range of defined pulse shapes has reported simulations showing that 20 μsec may be optimal to induce nerve activation using rectangular pulses. The strength (coil current) duration relationship is J-shaped, where at durations less than the optimum, the requisite current increases sharply, while at durations greater than the optimum, the needed current increases slowly. Differences between these results and those of Peterchev et al. 2013 may be that peripheral nerves, particularly motor neurons, are larger and easier to stimulate than thinner cortical axons. Notwithstanding, it is promising that the amplitudes achieved at pulse duration 23 μsec may sufficient or even optimal to achieve ulnar nerve excitation when pulses are applied directly over the distal part of the nerve and its distal branches.

The conventional biphasic waveform and the largely monophasic waveform achieved in this work share the characteristic that the main part of the pulse that is generally considered to produce the neural excitation is preceded by a period where the electric field is opposite in sign. It is not known for the general case whether the early part of the pulse partially cancels the later (and presumed active) part of the pulse, and whether this depends on the time scale of the waveform. The studies by Rapp et al. concluded that there were differences in threshold that appeared according to the waveform, with typical variations of on the order of a factor of 2× or less, but with different values according to the theoretical neuron model used.

In consideration of overall system efficiency, the extent to which the coil energy is recycled has a substantial effect. The non-recycled energy, in turn, is dependent on the integral of R Ic(t)² over the duration of the pulse, where R is the effective composite resistance seen by the coil current. For equivalent values of Ic, the new circuit described here has an integral approximately half of that of the conventional circuit, because of the shorter duration, which is favorable for achieving a high recycling fraction.

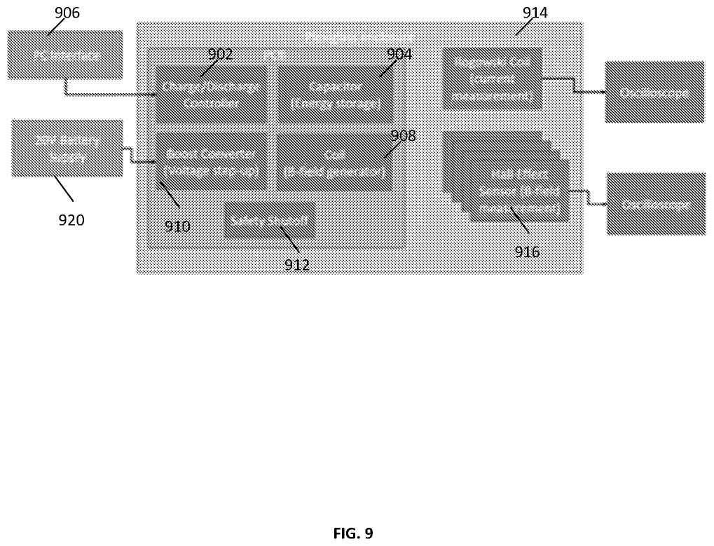

FIG. 9 depicts a block diagram of a portable rTMS system, in accordance with some embodiments. The system includes a controller 902 for controlling the charging and discharging of source capacitor Cs 904. The controller 902 may be coupled to a computer 906 or other type of processor-memory device, such that the computer may be used to vary the timing of the on and off of the switches, such as switches 160 and 143 of FIG. 1B, for example. The system may also include a boost converter 910 and a safety shutoff 912, a Rogowski coil 914 for current measurement and a Hall-Effect sensor 916 for magnetic field measurements. An oscilloscope may be used to measure and/or display the measurements of the Rogowski coil 914 and the Hall-Effect sensor 916.

In the example of FIG. 9, the source capacitor Cs 904 has a value of 380 uF, and is implemented with polypropylene dielectric to achieve low series resistance and high peak current. This capacitor is charged to a voltage in the range 100 to 300V using a pulsed boost system 910 from a primary power source 920 (e.g., a 20V battery or other type of power supply). The switch (which initiates the inductor charging and abruptly turns off its current) is implemented with a silicon IGBT, driven with a commercial gate driver, and is capable of handling voltages above 600V and achieving sub-micro turn on and turn off times. The return path of the inductor current is implemented with a high voltage, high current silicon diode and series resistor Rflyback. The series resistor is changed in the range 0.5 to 2 ohms, to produce the desired Lc/Rflyback time constant for the discharge of the inductor current. Since the resistor absorbs a significant fraction of the energy dissipated in each pulse, high current, high power resistors are used. Measurements are made of the generated magnetic field magnitude via Hall Effect magnetometers, and of the overall current flow with a Rogowski coil wrapped around one of the inductor legs. The coil (or inductor) 908 may be implemented as noted as a FIG. 8 shaped coil (e.g., 6 turns, radius 1.8 cm per side and inductance 6 uH) or as a circular coil (e.g., 8 turns, radius 2.6 cm, and inductance 7 uH), although other types of inductors or coils may be used as well.

In the example of FIG. 9, a printed circuit board may include the switch based on Si IGBTs with a maximum voltage of 1200V in parallel with a series combination of Si diodes that achieves >1200V voltage handling capability. The IGBTs and diodes are sized to allow current up to 2 KA in short (<100 tsec) pulses. Capacitors Cs and Cfly implemented with a polymer dielectric were chosen to assure low series resistance. Cs had the value 380 μF, and Cfly was varied over the range 3.8 μF to 11 μF to demonstrate control over the pulse duration and associated amplitude. The initial characteristic voltage Vo was varied up to a maximum of 300V using an external supply (although an on-board boost converter can provide this voltage starting from a 20V battery, for example). Although the previous example describes the switch as at least one insulated-gate bipolar transistors (IGBT), other types of switches such as one or more field effect transistors and one or more metal-oxide-semiconductor field-effect transistor, may be used as well.

Referring again to FIG. 1B, the transcranial magnetic stimulation system may include an inductor 150. This inductor may be disposed on, or proximate to, a surface of a head, such as the head of user 130. The inductor may generate a current that induces an electric field through electromagnetic induction. The inductor includes a first terminal and a second terminal. The first terminal is coupled in parallel to the charging unit 143, to a source resistor 152 and/or a source capacitor 158, and to fly back capacitor 164 (which may also be coupled to a fly back resistor 156). The second terminal may be coupled, via resistor 154, to the switch 160, to the fly back resistor 156 (which is coupled in series with the fly back capacitor 164), and diode 166. In the example of FIG. 1B, the switch 160 is configured to at least (1) close to discharge the at least one source capacitor towards the inductor to enable the current in the inductor to increase towards a threshold current amount and (2) open, in response to the threshold current amount through the inductor being reached, wherein energy from the inductor is transferred to an energy sink path (which in FIG. 1B comprises the fly back capacitor 164 and fly back resistor 156) thereby resulting in a reversal in the current through the inductor and a voltage pulse to be generated across the inductor.

Referring again to FIG. 1E, the transcranial magnetic stimulation system may include an inductor 150. This inductor may be disposed on, or proximate to, a surface of a head, such as the head of user 130. The inductor may generate a current that induces an electric field through electromagnetic induction. The inductor includes a first terminal and a second terminal. The first terminal is coupled in parallel to the charging unit 143, to a source capacitor 158, and to fly back capacitor 164. The second terminal may be coupled to the switch 160, to the fly back capacitor 164), and the diode 166. In the example of FIG. 1E, the switch 160 is configured to at least (1) close to discharge the at least one source capacitor towards the inductor to enable the current in the inductor to increase towards a threshold current amount and (2) open, in response to the threshold current amount through the inductor being reached, wherein energy from the inductor is transferred to an energy sink path (which in FIG. 1E comprises the fly back capacitor 164) thereby resulting in a reversal in the current through the inductor and a voltage pulse to be generated across the inductor.

Referring again to FIG. 1F, the transcranial magnetic stimulation system may include an inductor 150. This inductor may be disposed on, or proximate to, a surface of a head, such as the head of user 130. The inductor may generate a current that induces an electric field through electromagnetic induction. The inductor includes a first terminal and a second terminal. The first terminal is coupled in parallel to the charging unit 143, to a source capacitor 158, and to fly back capacitor 164. The second terminal may be coupled to the switch 160, to a fly back resistor 156 (which is coupled to the fly back capacitor 164), and the diode 166. In the example of FIG. 1F, the switch 160 is configured to at least (1) close to discharge the at least one source capacitor towards the inductor to enable the current in the inductor to increase towards a threshold current amount and (2) open, in response to the threshold current amount through the inductor being reached, wherein energy from the inductor is transferred to an energy sink path (which in FIG. 1F comprises the fly back capacitor 164 and fly back resistor 156) thereby resulting in a reversal in the current through the inductor and a voltage pulse to be generated across the inductor.

Referring again to FIG. 1G, the transcranial magnetic stimulation system may include an inductor 150. This inductor may be disposed on, or proximate to, a surface of a head, such as the head of user 130. The inductor may generate a current that induces an electric field through electromagnetic induction. The inductor includes a first terminal and a second terminal. The first terminal is coupled in parallel to the charging unit 143, to a source capacitor 158 (which may also include a source resistor), and to fly back capacitor 164 (which may also include a fly back resistor 156). The second terminal may be coupled to in parallel to the switch 160, to the fly back resistor 156 (which is coupled in series with the fly back capacitor 164), and diode 166. In the example of FIG. 1G, the switch 160 is configured to at least (1) close to discharge the at least one source capacitor towards the inductor to enable the current in the inductor to increase towards a threshold current amount and (2) open, in response to the threshold current amount through the inductor being reached, wherein energy from the inductor is transferred to an energy sink path (which in FIG. 1G comprises the fly back capacitor 164) thereby resulting in a reversal in the current through the inductor and a voltage pulse to be generated across the inductor.

Referring again to FIG. 4, the transcranial magnetic stimulation system may include an inductor 150. This inductor may be disposed on, or proximate to, a surface of a head, such as the head of user 130. The inductor may generate a current that induces an electric field through electromagnetic induction. The inductor includes a first terminal and a second terminal. The first terminal is coupled in parallel to the charging unit 143, to a source capacitor 158, and to fly back capacitor 408 (which may be coupled in parallel to a diode 406). The second terminal may be coupled to in parallel to the switch 160, to the fly back resistor 156 (which is coupled in series with the parallel combination of the fly back capacitor 408 and diode 406). In the example of FIG. 4, the switch 160 is configured to at least (1) close to discharge the at least one source capacitor towards the inductor to enable the current in the inductor to increase towards a threshold current amount and (2) open, in response to the threshold current amount through the inductor being reached, wherein energy from the inductor is transferred to an energy sink path (which in FIG. 4 comprises the fly back resistor 156 (which is coupled in series with the parallel combination of the fly back capacitor 408 and diode 406 thereby resulting in a reversal in the current through the inductor and a voltage pulse to be generated across the inductor.

The examples depicted at FIGS. 1B, 1E, 2F, 1G, and 4 are examples and may be modified based on the teachings of each of the circuits depicted, such that an aspect of one circuit may be utilized in combination with an aspect of another circuit. For example, the diode 166 of FIG. 1B may be added to FIG. 4, without departing from the disclosure herein.

Referring again to FIG. 1A and 1B as an example, there may be provided a method. The method may include placing an applicator, such as wand 120 which includes an inductor 150 disposed on, or proximate to, a surface of a head. The inductor generates a current that induces an electric field through electromagnetic induction, and the inductor includes a first terminal and a second terminal.

The method may also include initiating transcranial magnetic stimulation. For example, an operator of the transcranial magnetic stimulation system may trigger the pulse generation circuit 199 to generate one or more pulses which induce the electric field through electromagnetic induction. As noted, the transcranial magnetic stimulation system may include at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in at least one of: a reversal in the current through the inductor and a voltage pulse to be generated across the inductor or a drop in inductor current towards zero at a rate that is faster, when compared to a rate at which the current increases in the inductor while the switch is closed.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:
Example 1: A transcranial magnetic stimulation system comprising: an inductor configured to be disposed on, or proximate to, a surface of a head to generate a current that induces an electric field through electromagnetic induction, wherein the inductor includes a first terminal and a second terminal; at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in a reversal in the current through the inductor and a voltage pulse to be generated across the inductor.
Example 2: The transcranial magnetic stimulation system of Example 1, wherein while the switch is open, the reversal in current reverses at a rate that faster, when compared to a rate at which the current increases in the inductor while the switch is closed.
Example 3: The transcranial magnetic stimulation system of Examples 1-2, wherein the current reverses at the rate that is at least three times faster, when compared to the rate at which the current increases in the inductor while the switch is closed.
Example 4: The transcranial magnetic stimulation system of Examples 1-3, further comprising: a fly back capacitor coupled to at one end to the first terminal of the inductor and at the other end to the second terminal of the inductor, wherein the energy sink path comprises the fly back capacitor.
Example 5: The transcranial magnetic stimulation system of Examples 1-4, wherein the energy sink path provides a path for a rapid transfer of the energy during the reversal in the current through the inductor, and wherein the rapid transfer is caused in part by the fly back capacitor in the energy sink path, the fly back capacitor having a capacitance that smaller than a capacitance of the at least one source capacitor.
Example 6: The transcranial magnetic stimulation system of Examples 1-5, wherein the capacitance of the fly back capacitor is at least nine times smaller than the capacitance of the at least one source capacitor.
Example 7: The transcranial magnetic stimulation system of Examples 1-6, further comprising: a fly back resistor coupled to at one end to the second terminal of the inductor, wherein the energy sink path comprises the fly back resistor.
Example 8: The transcranial magnetic stimulation system of Examples 1-7, wherein the fly back resistor is further coupled to the first terminal of the inductor.
Example 9: The transcranial magnetic stimulation system of Examples 1-8, wherein the fly back resistor is further coupled to a fly back capacitor coupled to at one end to the first terminal of the inductor and at the other end to the second terminal of the inductor.
Example 10: The transcranial magnetic stimulation system of Examples 1-9, wherein the fly back capacitor is further coupled to a diode that is in parallel to the fly back capacitor.
Example 11: The transcranial magnetic stimulation system of Examples 1-10, wherein the at least one source capacitor is further coupled to a first terminal of the switch, and wherein the second terminal of the inductor is further coupled to a second terminal of the switch.
Example 12: The transcranial magnetic stimulation system of Examples 1-11, wherein the switch comprises at least one of: one or more insulated-gate bipolar transistors, one or more field effect transistors, and one or more metal oxide semiconductor field-effect transistor.
Example 13: The transcranial magnetic stimulation system of Examples 1-12, further comprising: a switch controller coupled to the switch to close the switch to enable the current in the inductor to increase towards a threshold current amount and, in response to the threshold current amount through the inductor being reached, open the switch.
Example 14: The transcranial magnetic stimulation system of Examples 1-12, wherein the threshold current amount being reached is determined based on an expiry of a timer or based on measurement of the current through the inductor reaching the threshold current amount.
Example 15: The transcranial magnetic stimulation system of Examples 1-14, further comprising: a diode coupled to the second terminal of the capacitor, the energy sink path, and a first terminal of the switch, wherein when the switch is open, the diode enables a portion of the reversal in current to be recycled into the at least one source capacitor.
Example 16: The transcranial magnetic stimulation system of Examples 1-15, further comprising: a charging unit providing a direct current power source to the at least one capacitor during a charging phase of the at least one capacitor.
Example 17: A transcranial magnetic stimulation system comprising: an inductor configured to be disposed on or proximate to a surface of a head to generate a current that induces an electric field through electromagnetic induction, wherein the inductor includes a first terminal and a second terminal; at least one source capacitor coupled to at least the first terminal of the inductor; and a switch configured to at least: close to discharge the at least one source capacitor towards the inductor; and open, in response to a threshold current amount through the inductor being reached, wherein energy from the inductor is transferred to an energy sink path thereby resulting in a drop in inductor current towards zero at a rate that is faster, when compared to a rate at which the current increases in the inductor while the switch is closed.
Example 18: The transcranial magnetic stimulation system of Example 17, wherein the current drops at the rate that is at least three times faster, when compared to the rate at which the current increases in the inductor while the switch is closed.
Example 19: The transcranial magnetic stimulation system of Example 17-18, wherein the energy sink path comprises at least one resistor coupled to the second terminal of the inductor.
Example 19: The transcranial magnetic stimulation system of Example 17-19, wherein the system further comprises one or more of the aspects claimed at any one or more of Examples 1-15.
Example 20: A method comprising: placing an applicator including an inductor disposed on, or proximate to, a surface of a head, wherein the inductor generates a current that induces an electric field through electromagnetic induction, wherein the inductor includes a first terminal and a second terminal; and initiating transcranial magnetic stimulation, wherein the inductor is comprised in a transcranial magnetic stimulation system, wherein the transcranial magnetic stimulation system comprises: at least one source capacitor coupled to at least the first terminal of the inductor; and a switch configured to at least: close to discharge the at least one source capacitor towards the inductor to enable the current in the inductor to increase towards a threshold current amount; and open, in response to the threshold current amount through the inductor being reached, wherein energy from the inductor is transferred to an energy sink path thereby resulting in at least one of: a reversal in the current through the inductor and a voltage pulse to be generated across the inductor or a drop in inductor current towards zero at a rate that is faster, when compared to a rate at which the current increases in the inductor while the switch is closed.
Example 21: The method of Example 21, wherein the method further comprises one or more of the aspects claimed at any one or more of Examples 1-19.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A transcranial magnetic stimulation system comprising:
an inductor configured to be disposed on, or proximate to, a surface of a head to generate a current that induces an electric field through electromagnetic induction, wherein the inductor includes a first terminal and a second terminal;
at least one source capacitor coupled to at least the first terminal of the inductor; and
a switch configured to at least:
close to discharge the at least one source capacitor towards the inductor to enable the current in the inductor to increase towards a threshold current amount; and
open, in response to the threshold current amount through the inductor being reached, wherein energy from the inductor is transferred to an energy sink path thereby resulting in a reversal in the current through the inductor and a voltage pulse to be generated across the inductor.

2. The transcranial magnetic stimulation system of claim 1, wherein while the switch is open, the reversal in current reverses at a rate that faster, when compared to a rate at which the current increases in the inductor while the switch is closed.

3. The transcranial magnetic stimulation system of claim 2, wherein the current reverses at the rate that is at least three times faster, when compared to the rate at which the current increases in the inductor while the switch is closed.

4. The transcranial magnetic stimulation system of claim 3, further comprising:
a fly back capacitor coupled to at one end to the first terminal of the inductor and at the other end to the second terminal of the inductor, wherein the energy sink path comprises the fly back capacitor.

5. The transcranial magnetic stimulation system of claim 4, wherein the energy sink path provides a path for a rapid transfer of the energy during the reversal in the current through the inductor, and wherein the rapid transfer is caused in part by the fly back capacitor in the energy sink path, the fly back capacitor having a capacitance that smaller than a capacitance of the at least one source capacitor.

6. The transcranial magnetic stimulation system of claim 5, wherein the capacitance of the fly back capacitor is at least nine times smaller than the capacitance of the at least one source capacitor.

7. The transcranial magnetic stimulation system of claim 1, further comprising:
a fly back resistor coupled to at one end to the second terminal of the inductor, wherein the energy sink path comprises the fly back resistor.

8. The transcranial magnetic stimulation system of claim 7, wherein the fly back resistor is further coupled to the first terminal of the inductor.

9. The transcranial magnetic stimulation system of claim 7, wherein the fly back resistor is further coupled to a fly back capacitor coupled to at one end to the first terminal of the inductor and at the other end to the second terminal of the inductor.

10. The transcranial magnetic stimulation system of claim 9, wherein the fly back capacitor is further coupled to a diode that is in parallel to the fly back capacitor.

11. The transcranial magnetic stimulation system of claim 1, wherein the at least one source capacitor is further coupled to a first terminal of the switch, and wherein the second terminal of the inductor is further coupled to a second terminal of the switch.

12. The transcranial magnetic stimulation system of claim 1, wherein the switch comprises at least one of: one or more insulated-gate bipolar transistors, one or more field effect transistors, and one or more metal-oxide-semiconductor field-effect transistor.

13. The transcranial magnetic stimulation system of claim 1, further comprising:
a switch controller coupled to the switch to close the switch to enable the current in the inductor to increase towards the threshold current amount and, in response to the threshold current amount through the inductor being reached, open the switch.

14. The transcranial magnetic stimulation system of claim 1, wherein the threshold current amount being reached is determined based on an expiry of a timer or based on measurement of the current through the inductor reaching the threshold current amount.

15. The transcranial magnetic stimulation system of claim 1, further comprising:
a diode coupled to the second terminal of the capacitor, the energy sink path, and a first terminal of the switch, wherein when the switch is open, the diode enables a portion of the reversal in current to be recycled into the at least one source capacitor.

16. The transcranial magnetic stimulation system of claim 1, further comprising:
a charging unit providing a direct current power source to the at least one capacitor during a charging phase of the at least one capacitor.

17. A transcranial magnetic stimulation system comprising:
an inductor configured to be disposed on or proximate to a surface of a head to generate a current that induces an electric field through electromagnetic induction, wherein the inductor includes a first terminal and a second terminal;
at least one source capacitor coupled to at least the first terminal of the inductor; and
a switch configured to at least:
close to discharge the at least one source capacitor towards the inductor; and
open, in response to a threshold current amount through the inductor being reached, wherein energy from the inductor is transferred to an energy sink path thereby resulting in a drop in inductor current towards zero at a rate that is faster, when compared to a rate at which the current increases in the inductor while the switch is closed.

18. The transcranial magnetic stimulation system of claim 17, wherein the current drops at the rate that is at least three times faster, when compared to the rate at which the current increases in the inductor while the switch is closed.

19. The transcranial magnetic stimulation system of claim 17, wherein the energy sink path comprises at least one resistor coupled to the second terminal of the inductor.

20. A method comprising:
placing an applicator including an inductor disposed on, or proximate to, a surface of a head, wherein the inductor generates a current that induces an electric field through electromagnetic induction, wherein the inductor includes a first terminal and a second terminal; and
initiating transcranial magnetic stimulation, wherein the inductor is comprised in a transcranial magnetic stimulation system, wherein the transcranial magnetic stimulation system comprises:
at least one source capacitor coupled to at least the first terminal of the inductor; and a switch configured to at least:
close to discharge the at least one source capacitor towards the inductor to enable the current in the inductor to increase towards a threshold current amount; and
open, in response to the threshold current amount through the inductor being reached, wherein energy from the inductor is transferred to an energy sink path thereby resulting in at least one of:
a reversal in the current through the inductor and a voltage pulse to be generated across the inductor, or
a drop in inductor current towards zero at a rate that is faster, when compared to a rate at which the current increases in the inductor while the switch is closed.
